SEQUENCE LISTING (11 sequences)

SEQ ID NO: 1 - DNA, 1442 bp, Streptomyces Beta-Vulgaris
ttaacacatgcaagtcgaacgatgaagccgcttcggtggtggattagtggcgaacgggtgagtaacacgtgggcaatctgccctgcactctgggacaagccctggaaacggggtctaataccggatatgactaccgatcgcatggttggtggtggaaagctccggcggtgcaggatgagcccgcggcctatcagcttgttggtggggtgatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgagagtgacggtacctgcagaagaagcgccggctaactacgtgccagcagccgcggtaatacgtagggcgcgagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacaccggaaacggccagagatggtcgcccccttgtggtcggtgtacaggtggtgcatggctgtcgtcagctcgtgtcgtgagatgttgggttaagtcccgcaacgagcgcaacccttgttctgtgttgccagcatgcctttcggggtgatggggactcacaggagactgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttggggagggagctgtcgaaggtgggactggcgattgggacgaagtcgtaacaag

SEQ ID NO: 2 - DNA, 20 bp, Artificial Sequence
  Description of the Artificial Sequence Universal Primer 27 f
agagtttgatcctggctcag

SEQ ID NO: 3 - DNA, 19 bp, Artificial Sequence
  Description of the Artificial Sequence Universal Primer 1492 r
ggttaccttgttacgactt

SEQ ID NO: 4 - DNA, 24 bp, Artificial Sequence
  Description of the Artificial Sequence  Primer PR1 f
ctggctattctcgatttttaatcg

SEQ ID NO: 5 - DNA, 24 bp, Artificial Sequence
  Description of the Artificial Sequence  Primer PR1 r
tcctgcatatgatgctccttattg

SEQ ID NO: 6 - DNA, 30 bp, Artificial Sequence
  Description of the Artificial Sequence  Primer PDF1 f
tcatggctaagtttgcttccatcatcaccc

SEQ ID NO: 7 - DNA, 24 bp, Artificial Sequence
  Description of the Artificial Sequence  Primer PDF1 r
gtagatttaacatgggactgcgac

SEQ ID NO: 8 - DNA, 21 bp, Artificial Sequence
  Description of the Artificial Sequence  Primer PAL1 f
cggaggaggagtggacgctat

SEQ ID NO: 9 - DNA, 21 bp, Artificial Sequence
  Description of the Artificial Sequence  Primer PAL1 r
tgcgacaccgtttttggttct

SEQ ID NO: 10 - DNA, 24 bp, Artificial Sequence
  Description of the Artificial Sequence  Prime r EF1 f
atgccccaggacatcgtgatttca

SEQ ID NO: 11 - DNA, 24 bp, Artificial Sequence
  Description of the Artificial Sequence  Primer EF1 r
ttggcggcacccttagctggatca

(19) United States
(12) Patent Application Publication
(10) Pub. No.: US 2011/0143940 A1
(43) Pub. Date: Jun. 16, 2011

(54) STREPTOMYCES BETA-VULGARIS STRAIN, CULTURE FILTRATE, DERIVED ACTIVE COMPOUNDS AND USE THEREOF IN THE TREATMENT OF PLANTS

(75) Inventors: Ahmed Lebrihi, Noueilles (FR); Rafik Errakhi, Benguerir (MA); Mustapha Barakate, Marrakech (MA)

(73) Assignees: AGRONUTRITION, Carbonne (FR); INSTITUT NATIONAL POLYTECHNIQUE DE TOULOUSE, Toulouse (FR)

(21) Appl. No.: 13/000,055

(22) PCT Filed: Jun. 19, 2009
(86) PCT No.: PCT/FR2009/051181

§ 371 Date: Feb. 2, 2011

(30) Foreign Application Priority Data
Jun. 19, 2008 (FR) 08 54051

(51) Int. Cl. A01N 63/00; C12N 1/20; C12P 21/00; A01P 21/00; A01P 3/00

(52) U.S. Cl. 504/117; 435/253.5; 435/71.3; 424/93.43; 424/234.1

(57) ABSTRACT

A new Streptomyces beta-vulgaris strain that is deposited at the CNCM under number I-3639, and the use of this strain or a mutant strain for obtaining a composition (culture filtrate) and active compounds (secondary metabolites) by fermentation. Also, the use of the new strain, a mutant strain, the culture filtrate, or active compounds for the protection of plants and the biological struggle against phytopathogenic agents and/or for stimulating the growth of plants.

This invention has as its object a new actinomycete Streptomyces beta-vulgaris strain or a mutant strain of the latter.

The purpose of the invention is also the use of this strain for producing a composition, or culture filtrate, by fermentation and active compounds.

The invention also relates to the use of this beta-vulgaris strain, a mutant strain, culture filtrate, or an active compound for stimulating the growth of plants and/or for fighting against infections caused by phytopathogenic agents.

For several decades, the agricultural world has sought new solutions for improving the quality of soils and for stimulating plant growth.

For this purpose, numerous products have been developed for fertilizing the soil, stimulating nutrition and therefore the growth of cultivated plants.

However, these products are generally essentially of chemical origin and are not satisfactory from the environmental and human standpoint, although there is a need for substances of natural origin that are capable of effectively stimulating the growth of plants.

In addition, the plants are fragile and are able to be infected by various factors, in particular by phytopathogenic agents, which consequently reduce the agricultural quality and yield. Among the pathogenic agents, the fungi, responsible for fungal illnesses, are those that have the greatest economical impact. Every plant strain is sensitive to one or more primary illnesses that are able to greatly reduce its strength, its growth, and finally the quantity and/or the quality of the harvest.

It is known that the phytogenic illnesses reduce the world agriculture production by 12 to 14%, whereby 70% of the damage is of fungal origin. It is therefore essential to find effective means for fighting against the infections that are caused by phytopathogenic agents.

Currently, several fighting means are employed.

A first means, simple to implement, consists in using farming techniques such as plowing or crop rotation. However, the pathogenic agents produce sclerotes in the soil that make it possible for them to survive for several years, although the farming practices remain ineffective in the absence of means eradicating in the soil the forms of resistance and the pathogenic mycelium.

A second method, which is the most used, consists in chemically treating the infected crops. For example, the methyl bromide treatment, which is the most used chemical fungicide, generally for disinfecting the soil before sowing the crops, is known.

However, the chemical treatment is no longer satisfactory because the use of chemical products causes negative effects on the environment and on human health. Methyl bromide in particular constitutes a threat for the stratosphere because of the catalytic power of the bromine atom.

In addition, the effectiveness of chemical agents is increasingly limited because of the appearance of resistant pathogenic strains.

For several years, solutions that are more effective and more respectful of the environment have therefore been sought. The specialists are oriented toward the biological struggle based on the use of natural antagonistic biological agents.

Several microorganisms, such as Trichoderma harzianum, Trichoderma koningii or Pseudomonas fluorescens, have been used for controlling phytopathogenic illnesses.

However, the existing biological agents are not satisfactory in terms of effectiveness and reliability, and there is a need for natural biological agents that are non-toxic for man and the environment, able to protect the plants effectively by stimulating their growth and/or by fighting against the phytopathogenic illnesses.

This is why the purpose of this invention is to meet this need by proposing a new bacterial strain that is particularly effective for stimulating the growth of plants and/or for fighting against phytopathogenic infections.

For this purpose, the invention has as its object an actinomycete Streptomyces beta-vulgaris strain that is deposited at the National Collection of Microorganism Cultures CNCM under number 1-3639 and at the Belgian Microorganism Collection Center LGM [Methodological Laboratory of the Geographical Sciences of the University of Liege] under number P-23735, or a mutant strain, characterized by a sequence of the DNAr 16S SEQ No. 1 that is deposited in the nucleotide databank Pubmed/NCBI, under the reference: DQ303238.

In the following description of the invention, this strain can be named “I-3639.”

Advantageously, this strain is very effective both for stimulating the growth of plants and for fighting against the phytopathogenic agents.

Advantageously, this strain is very effective in the biological struggle against the phytopathogenic agents. It is also effective for the stimulation of the growth of plants, in particular by improving the assimilation of nutrients by the plants.

The invention also relates to the use of the strain I-3639 or a mutant strain for obtaining—by fermentation—a culture filtrate comprising secondary metabolites, having an activity that is anti-bacterial, anti-fungal, and that stimulates the growth of plants.

The purpose of this invention is also the use of the strain I-3639 or a mutant strain for obtaining metabolites or active compounds, in particular antibiotics.

The purpose of the invention is also the use of the I-3639 strain or a mutant strain of the culture filtrate that is obtained by fermentation of the strain, or active compounds that are obtained by fermentation of the strain, for the treatment of plants, in particular for the struggle against phytopathogenic infections and/or for promoting the growth of plants. In particular, it involves the use:
As an anti-bacterial agent for the protection of plants and the biological struggle against phytopathogenic agents, for example for the protection of plants relative to gram-positive bacteria, and/or
As an antifungal agent for the protection of plants and the biological struggle against phytopathogenic agents, and/or
For activating natural defense systems of plants, in particular for stimulating the expression of defense genes of the plants PR1 and PDF1.2a, and/or
For stimulating the growth of plants, and/or
For improving the assimilation of nutrients by the plants, in particular for improving the assimilation of potassium, zinc, boron, calcium, copper, phosphorus, magnesium, iron, manganese, and nitrogen.

The invention is now described in detail, with regard to the accompanying drawings in which:

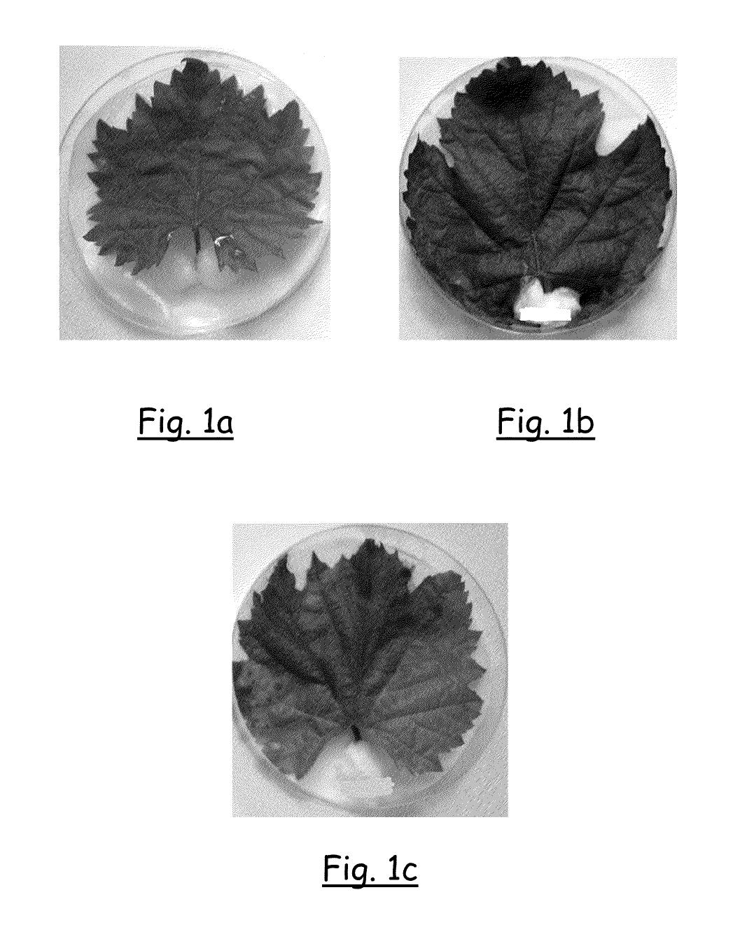

FIG. 1a shows a photo of a leaf of a vine without treatment (control),

FIG. 1b shows a photo of a leaf of a vine that is treated by Botrytis cinerea and then by the culture filtrate that is obtained by fermentation of the strain I-3639 according to the invention, and

FIG. 1c shows a photo of a leaf of a vine that is treated by Botrytis cinerea.

### I. STRAIN ISOLATION PROTOCOL ACCORDING TO THE INVENTION

The beta-vulgaris I-3639 strain according to the invention is isolated according to a selection process that comprises the following stages:
Bringing a biological sample that can contain the beta-vulgaris strain and/or a mutant strain into the presence of a suitable medium for the selection of actinomycete strains,
Treatment of said sample for isolating the actinomycete strains that are present, and
Isolating the beta-vulgaris strain and/or a mutant strain based on its capacity to inhibit phytopathogenic agents.

The isolation of the strain according to the invention can be carried out starting from samples of the sugar beet rhizosphere soil of Beni-Mellal in Morocco.

The protocol is described as follows.

Soil samples of a sugar beet rhizosphere soil of Beni-Mellal are taken with an auger of more than 10 cm of depth, after having removed about 3 cm of the surface of the soil.

The samples that are taken are placed in sterile polyethylene packets, sealed and stored at 4° C.

The actinomycete strains are then isolated in an OL (Olson) medium or an SEA (Soil Extract Agar) medium according to two methods: treatment by heat-stirring or treatment by chemotactism.

Preferably, the samples are treated by the heat-stirring method.

The samples are mixed, put into suspension in sterile distilled water (4 g per 36 ml) and stirred at 200 rpm for 30 minutes. They are then treated at 50° C. for 10 minutes.

Each sample is then diluted at 10⁻⁷, dispersed (0.1 ml) on the surface of an OL medium or an SEA medium, preferably SEA, and then incubated at 30° C. for 21 days.

According to one variant, the samples can be treated by chemotactism.

Antibiotics are added to the isolation medium (OL and SEA), such as nalixidic acid (10 mg·L⁻¹) and novobiocin (25 mg·L⁻¹). Preferably, nalixidic acid is added. Cycloheximide anti-fungal compounds (40 mg·L⁻¹) can also be added to the medium.

The samples are then incubated at 30° C. for 3 weeks.

The treatment of the samples can be preceded by a pretreatment stage by, for example, heating, microwaves, electrical pulses or else using chemical reagents.

After the samples are treated, the actinomycete strains are isolated by recognition in a microscope based on their morphological characteristics.

The actinomycetes are transferred onto a Bennett medium. The pure isolates are kept at 4° C. for 2 months. Alternatively, the cultures are resuspended and preserved in 20% sterile glycerol at −20° C.

To isolate specifically the strain that is the object of this invention, a screening based on its capacity to inhibit the following pathogenic agents is done:
Sclerotium rolfsii
Fusarium oxysporum
Verticillium dahliae
Botrytis cinerea
Pythium ultimum

The selection is carried out by agar diffusion with the method of cylinders (Bauer et al., 1966). Pure actinomycetes are inoculated on a Bennett medium. After a 5-day incubation at 30° C., a calibrated cylinder (6 mm in diameter) is cut out in the culture medium and placed on a test medium that contains tested phytopathogenic agents. The culture medium is a PDA medium for the pathogenic fungi and a Bennett medium for the pathogenic bacteria.

The strains are then kept at 4° C. for 4 hours to make possible the diffusion of antibiotic compounds and then incubated at 30° C. The diameters of inhibition are determined after 48 hours of incubation.

Among the different strains that show a capacity for inhibition of the tested pathogenic agents, the I-3639 strain according to the invention is selected for its capacity for inhibition of the sclerotes of Sclerotium rolfsii and the hyphal growth of Sclerotium rolfsii.

For the germination test of sclerotes, sclerotes of Sclerotium rolfsii are collected and disinfected with 2% sodium hypochlorite for 3 minutes and washed 3 times with sterile distilled water. Ten sclerotes were placed on the surface of an agar substrate that contains a PDA medium that is mixed with the actinomycete strains, and the number of germinated sclerotes is counted after 72 hours of incubation at 25° C.

For the hyphal growth test, an 8 mm mycelium disk was sampled from 5-day colonies of Sclerotium rolfsii and transferred onto a substrate. The mycelium growths are evaluated by measuring the diameter of colonies after 72 hours of incubation at 25° C.

The strain according to the invention is selected for its capacity for inhibition of the sclerotes of Sclerotium rolfsii of 71% and the hyphal growth of Sclerotium rolfsii of 80%. It can be recognized by these phenotypical and physiological characteristics that are described below.

### II. CHARACTERIZATION OF THE STRAIN ACCORDING TO THE INVENTION

The new beta-vulgaris strain, object of the invention, is a bacterial strain that belongs to the family of actinomycetes and to the genus of Streptomyces. It was deposited on Jun. 30, 2006 at the CNCM under number I-3639 and at the Belgian Microorganism Collection Center LGM under number P-23735.

The actinomycetes or the actinobacteria are gram-positive bacteria that have a filamentous form. These are saprophyte bacteria that are capable of degrading the organic material in the soil and of using more complex molecules for their growth.

The beta-vulgaris bacterial strain according to the invention is characterized by a whitish-yellow aerial mycelium on an ISP2 medium or Bennett agar. The substrate mycelium varies from brownish-yellow to brownish-gray according to the culture medium.

The I-3639 strain has smooth spores with S-type spiral chains. The chains comprise 10 to 50 spores on average.

The growth temperature is between 12 and 37° C., preferably between 28 and 32° C. The pH of the culture medium is preferably between 5 and 9.

For its growth, the strain is to be cultivated in a medium that contains a variety of nutrients. For example, as a carbon source, it is possible to use D-glucose, D-fructose, D-maltose, D-lactose, L-rhamnose, and dextrin. By contrast, it is preferable not to use L-arabinose, raffinose, sorbitol, D-galactose and cellulose.

As a nitrogen source, it is possible to use amino acids, nitrate salts, and ammonium salts.

The strain according to the invention does not hydrolyze the starch. It reduces the nitrates into nitrites and degrades the adenine, Tween 20, and sodium acetate.

The strain according to the invention can also be characterized by its molecular characteristics. The primary molecular analyses used for the determination of the bacterial strains are the sequencing of ribosomal DNA (DNAr) 16S and the DNA-DNA hybridization.

The DNA-DNA hybridization shows that the I-3639 strain according to the invention corresponds to a new strain in the Streptomyces group.

The sequencing of the DNAr 16S consists in extracting the genomic DNA and then in amplifying by PCR the DNAr 16S portion by using specific primers. The product of the PCR is then used for sequencing the portion of the DNAr 16S.

The amplification of the DNAr 16S portion of the bacterial strain according to the invention has been done by using two universal 27f primers of the sequence SEQ No. 2 (5′-AGAGTTTGATCCTGGCTCAG-3′) and 1492r of the sequence SEQ No. 3 (5′-GGTTACCTTGTTACGACTT-3′). The DNAr 16S sequence that characterizes the bacterial strain according to the invention is the sequence SEQ No. 1:

| | |
|---|---|
| 1 | ttaacacatg caagtcgaac gatgaagccg cttcggtggt ggattagtgg cgaacgggtg |
| 61 | agtaacacgt gggcaatctg ccctgcactc tgggacaagc cctggaaacg gggtctaata |
| 121 | ccggatatga ctaccgatcg catggttggt ggtggaaagc tccggcggtg caggatgagc |
| 181 | ccgcggccta tcagcttgtt ggtggggtga tggcctacca aggcgacgac gggtagccgg |
| 241 | cctgagaggg cgaccggcca cactgggact gagacacggc ccagactcct acgggaggca |
| 301 | gcagtgggga atattgcaca atgggcgaaa gcctgatgca gcgacgccgc gtgagggatg |
| 361 | acggccttcg ggttgtaaac ctctttcagc agggaagaag cgagagtgac ggtacctgca |
| 421 | gaagaagcgc cggctaacta cgtgccagca gccgcggtaa tacgtagggc gcgagcgttg |
| 481 | tccggaatta ttgggcgtaa agagctcgta ggcggcttgt cacgtcggat gtgaaagccc |
| 541 | ggggcttaac cccgggtctg cattcgatac gggcaggcta gagttcggta ggggagatcg |
| 601 | gaattcctgg tgtagcggtg aaatgcgcag atatcaggag gaacaccggt ggcgaaggcg |
| 661 | gatctctggg ccgatactga cgctgaggag cgaaagcgtg gggagcgaac aggattagat |
| 721 | accctggtag tccacgccgt aaacgttggg aactaggtgt gggcgacatt ccacgtcgtc |
| 781 | cgtgccgcag ctaacgcatt aagttccccg cctggggagt acggccgcaa ggctaaaact |
| 841 | caaaggaatt gacgggggcc cgcacaagcg gcggagcatg tggcttaatt cgacgcaacg |
| 901 | cgaagaacct taccaaggct tgacatacac cggaaacggc cagagatggt cgcccccttg |
| 961 | tggtcggtgt acaggtggtg catggctgtc gtcagctcgt gtcgtgagat gttgggttaa |
| 1021 | gtcccgcaac gagcgcaacc cttgttctgt gttgccagca tgcctttcgg ggtgatgggg |
| 1081 | actcacagga gactgccggg gtcaactcgg aggaaggtgg ggacgacgtc aagtcatcat |
| 1141 | gccccttatg tcttgggctg cacacgtgct acaatggccg gtacaatgag ctgcgatacc |
| 1201 | gtgaggtgga gcgaatctca aaaagccggt ctcagttcgg attggggtct gcaactcgac |
| 1261 | cccatgaagt cggagtcgct agtaatcgca gatcagcatt gctgcggtga atacgttccc |
| 1321 | gggccttgta cacaccgccc gtcacgtcac gaaagtcggt aacacccgaa gccggtggcc |
| 1381 | caaccccttg gggagggagc tgtcgaaggt gggactggcg attgggacga agtcgtaaca |
| 1441 | ag |

This sequence SEQ No. 1 is deposited in the nucleotide databank Pubmed/NCBI, under the reference: DQ303238.

The purpose of this invention is also the mutant strains of the beta-vulgaris I-3639 strain.

“Mutant strain” is intended to designate any strain that can be obtained by selective mutation from the beta-vulgaris I-3639 strain.

The mutant strains according to the invention are obtained by putting the beta-vulgaris I-3639 strain into the presence of a physical mutagenic agent, such as radiation, or a chemical mutagenic agent, or different concentrations of nitrosoguanidine, and then in selecting—in a suitable medium—the remaining advantageous mutants by using their antibiotic spectrum.

Advantageously, the mutant strains that are obtained have production capacities of certain metabolites that are 10 to 100 times higher than those of the strain that is not mutated according to the invention.

The beta-vulgaris I-3639 strain according to the invention, or its mutant strains, show important biological activities relative to bacteria such as Micrococcus luteus, Bacillus subtilis, Bacillus cereus and Streptomyces scabies, and against phytopathogenic fungi such as Fusarium oxysporum f.sp. albedinis, Botrytis cinerea, Phythium ultimum, and Verticillium dahliae.

The beta-vulgaris Streptomyces strain according to the invention or its mutant strains also have a high activity against fungi that cause wood illnesses of the vine such as Phaeomoniella chlamydospora, Phaeomoniella aleophilum, Eutypa lata, Fomitiporia mediterranea and Botryosphaeria obtusa.

The spectrum of activities of the beta-vulgaris strain according to the invention relative to certain microorganisms is presented in the table below. Four degrees of activity have been measured:

| | Activity of the Strain |
|---|---|
| | According to the |
| Microorganism | Invention |
| Phaeomoniella chlamydospora | ++ |
| Phaeomoniella aleophilum | ++ |
| Fomitiporia mediterranea | ++ |
| Eutypa lata | +++ |
| Botryosphaeria obtusa | +++ |
| Botryosphaeria dothidea | +++ |
| Sclerotium rolfsii | +++ |
| Botrytis cinerea | +++ |
| Verticillium dahliae | +++ |
| Fusarium oxysporum | ++ |
| Pythium ultimum | ++ |
| Streptomyces scabies | +++ |
| Micrococcus luteus | +++ |
| Bacillus subtilis | +++ |
| Pseudomonas fluorescens | + |
| Escherichia coli | + |

− Span of activity
+ Activity (inhibition of 10 to 15 mm)
++ High activity (inhibition of 15 to 20 mm)
+++ Very high activity (inhibition of greater than 20 mm)

### III. CULTURE FILTRATE THAT CAN BE PRODUCED BY FERMENTATION OF THE STRAIN ACCORDING TO THE INVENTION

According to another aspect, the purpose of the invention is the use of the beta-vulgaris I-3639 strain or a mutant strain for obtaining a composition that comprises in particular metabolites by fermentation in a liquid medium.

The beta-vulgaris strain according to the invention can actually be used for producing—by fermentation—a composition, a culture filtrate, in particular containing secondary metabolites.

The culture filtrate is obtained by cultivation of the beta-vulgaris I-3639 strain in a liquid medium in a fermenter.

The production process is as follows:
Cultivation of beta-vulgaris bacteria according to the invention or a mutant strain in a fermenter at 30° C. for 10 days, in a liquid medium that consists of glucose, malt extract, yeast extract, and water,
Centrifuging of the mixture,
Filtration through a filter with pores that are 45 μm in diameter,
Recovery of the culture filtrate.

The culture filtrate is a composition that comprises elements of the culture medium and metabolites that are produced during fermentation.

The fermentation stage can be done according to the procedure described below.

A sample of the strain from a mature inclined culture is inoculated in an Erlenmeyer flask that contains 50 ml of the sterile culture medium that contains 0.4% glucose, 1% of a malt extract, and 0.4% of a yeast extract (adjusted to pH 7.2 before sterilization) and cultivated in a rotary stirring device (250 rpm) at 30° C. for 10 days.

The centrifuging and filtration stages can be implemented according to the procedure that is described below.

The nutrient broth (10 liters) is centrifuged at 12,000 rpm for 15 minutes, and the supernatant is extracted twice with an equal volume of ethyl acetate. The organic phase is dehydrated with Na₂SO₄ and dried with a rotary evaporator. The raw red extract that is obtained is suspended in a minimum volume of MeOH and then filtered through a filter with pores that are 45 μm in diameter.

The culture filtrate that is obtained from the fermentation of the strain according to the invention can be used for the struggle against the phytopathogenic agents and/or for stimulating the growth of plants.

### IV. ACTIVE COMPOUNDS THAT CAN BE PRODUCED BY FERMENTATION OF THE STRAIN ACCORDING TO THE INVENTION

According to another aspect, the purpose of the invention is the use of the beta-vulgaris I-3639 strain or a mutant strain for obtaining active compounds by fermentation in a liquid medium.

The beta-vulgaris strain according to the invention can actually be used for producing secondary metabolites, in particular antibiotics, by fermentation.

These active compounds can be obtained by:
Cultivation of the beta-vulgaris I-3639 strain or a mutant strain in a liquid medium in a fermenter,
Centrifuging,
Filtration,
And then purification.

The stages of fermentation, centrifuging and filtration are such as described in III.

The production of the total antibacterial activity is done on a nutrient agar by a diffusion test on agar against Bacillus subtilis ATCC 6633 and Mucor sp. The inhibition of the growth is examined after 24 hours of incubation at 30° C. The antimicrobial activity is evaluated by measuring the diameter of the inhibition zone.

To recover the active compounds that are produced by the strain according to the invention or a mutant strain and that are present in the culture filtrate, the filtration stage is followed by a stage for purification of the active compounds, which can be done as indicated below.

The filtrate is eluted in reverse-phase HPLC under the following conditions for obtaining pure products: Uptisphere column UP5ODB C18 (250×7 8 mm) subjected to a solvent system with a linear gradient of 0% to 100% of MeOH for 45 minutes and with an isocratic gradient at 100% MeOH in H₂O for 20 minutes, with a flow rate of 1 ml/minute and UV detection at 220 nm. The fractions that correspond to the active peaks are obtained by testing against Bacillus subtilis and Mucor ramanniamus.

The active compounds that are obtained are in particular secondary metabolites such as antibiotics that are involved in the biological struggle and the monitoring of phytopathogenic illnesses.

### V. USE OF A BETA-VULGARIS I-3639 STRAIN, A MUTANT STRAIN, CULTURE FILTRATE, OR AN ACTIVE COMPOUND

The strain according to the invention has important phytostimulating properties, although it can be used for stimulating the growth of plants.

In particular, the beta-vulgaris I-3639 strain is capable of stimulating the root portion of plants, by increasing the number of root absorbent hairs, and of stimulating the aerial portion as well as the flowering of plants. It is also capable of significantly improving the assimilation of nutrients by the plants.

It is the same for a mutant strain, the culture filtrate, or active compounds that are obtained by fermentation of the I-3639 strain, or a mutant strain.

In addition, the different studies that relate to the characteristics of the strain or a mutant strain, the culture filtrate, or active compounds that are produced by this strain, show that said strain (or mutant strain), said filtrate and said active compounds are very effective from the standpoint of their antibacterial and antifungal properties, which impart to them in particular the capacity for being used as agents of biological struggle.

The beta-vulgaris strain according to the invention, a mutant strain, the culture filtrate or an active compound produced from this strain, can therefore be used as an antibacterial and/or antifungal agent for the protection of the plants and the biological struggle against phytopathogenic agents.

They can be used specifically for the protection of plants relative to the gram-positive bacteria or else relative to the phytopathogenic fungi. Tests have been carried out to illustrate the action of the strain according to the invention, a mutant strain, the culture filtrate, and its active compounds on the plants.

1) Study of the Phytostimulating Properties

The purpose of this study is to show the capacity of the strain according to the invention and its active compounds in order to stimulate the growth of plants.

The study is done on Arabidospsis thaliana plantlets.

The plantlets are pretreated at the roots by the strain according to the invention, its culture filtrate or its active compounds that are obtained after fermentation and purification, and then are inoculated again on a Gamgorg B5 culture medium. The unit is incubated in a culture chamber at 25° C.

The synthesis of the reading of the results done after one week of incubation is shown in the table below:

| | | Treated with the Strain |
|---|---|---|
| | Control | According to the Invention |
| Weight (g) | 0.140 | 0.205 |
| Size of the Plants (mm) | 38 | 48 |
| Length of the Roots (mm) | 24 | 38 |

These results show well that the I-3639 strain according to the invention clearly stimulates the growth of plants, in particular the Arabidospsis thaliana plantlets.

In particular, it stimulates the root portion of the plants. It also makes it possible to increase their size and their weight in a considerable way.

The same study was done on vine vitro-plant plantlets.

The vitro-plants are pretreated at the roots by the strain according to the invention or by the culture filtrate or an active compound, and then are inoculated again on a Gamgorg B5 culture medium. The unit is incubated in a culture chamber at 25° C.

The reading of the results that is done after a week of incubation shows that the I-3639 strain clearly stimulates the growth of vitro-plants of the vine, in particular the root portion.

2) Improvement of the Assimilation of Nutrients by the Plants

This study has as its objective to evaluate the effect of the I-3639 strain, in particular the culture filtrate according to the invention, on the assimilation of nutrients by the plants.

The tests are conducted on the tender wheat.

The wheat seeds are sterilized by immersing them in a mixture of distilled water and bleach for 10 minutes. They are then rinsed three times in sterile distilled water and dried on Wattman paper under a hood.

The seeds are then planted in perforated plastic pots that each contain 500 g of soil. Each pot receives 10 seeds that are planted at a depth of 1 cm. The pots are watered daily.

After two weeks of growth, the leaves are treated by foliar spraying with the culture filtrate according to the invention (obtained by the implementation of the process at point III) or with water (control) or with the organomineral fertilizer (positive control). The filtrate is mixed with water. Different proportions have been tested (30% and 50%).

The tests are repeated five times for each test.

After three weeks of growth, the leaves are ground to 500 microns, and then dried in the oven at 105° C. until a constant weight is reached.

The assimilation of the nitrogen is measured by the Dumas method. For the other minerals, an analysis by atomic emission spectrometry is done. The results that are obtained are presented in the table below:

| | Nitrogen | Phosphorus | Potassium | Calcium | Magnesium | Boron | Copper | Iron | Manganese | Zinc |
|---|---|---|---|---|---|---|---|---|---|---|
| Control | 1.24 | 0.75 | 2.47 | 0.68 | 0.19 | 14 | 4.86 | 41.1 | 319 | 41.7 |
| (Water) | | | | | | | | | | |
| Organomineral | 1.74 | 0.54 | 2.27 | 0.74 | 0.25 | 17.3 | 4.63 | 232 | 1048 | 42.2 |
| Fertilizer | | | | | | | | | | |
| 30% Culture | 1.83 | 0.63 | 2.47 | 0.8 | 0.27 | 15.5 | 8.36 | 387 | 1840 | 53.6 |
| Filtrate | | | | | | | | | | |
| 50% Culture | 1.63 | 0.58 | 2.35 | 0.75 | 0.24 | 14.6 | 8.48 | 359 | 1332 | 48.8 |
| Filtrate | | | | | | | | | | |

The results for the total nitrogen, the total phosphorus, the total potassium, the total calcium, and the total magnesium are provided by percentage of dry material.

The results for the total boron, the total copper, the total iron, the total manganese, and the total zinc are provided in mg/kg of dry material.

It is noted that the invention makes it possible to significantly improve the assimilation of nutrients by the plants and therefore to promote their growth.

3) Study of the Antifungal and Antibacterial Properties

3-a) Study of the Protection of the Vine Against Illnesses of the Wood

This study has the objective of showing the capacity of the strain according to the invention, of the culture filtrate, and its active compounds to protect in vitro vine plants against illnesses of the wood (ESCA, ethypiosis, BDA) that are caused by phytopathogenic fungi.

The operating procedure is as follows.

Internal nodes of five-week vine vitro-plants (sauvignon variety of grape and colombard variety of grape) are cut, treated with spores of the I-3639 strain, and then inoculated on an MS medium under monitored conditions.

After 5 weeks of incubation, the leaves of each vitro-plant are treated by a suspension of Eutypia lata phytopathogenic fungal spores.

Reading the results is done after one week of infection.

The results that are obtained show that the I-3639 strain is able to protect the vine against phytopathogenic fungal infections.

3-b) Study of the Protection of the Vine Against Botrytis cinerea

This study has as its objective to show the capacity of the strain according to the invention to protect vine plants against Botrytis cinerea, phytopathogenic fungus responsible for gray rot, a cryptogamic illness that runs wild on several crops of major agronomical interest such as the vine, the sunflower, or the tomato.

The tests are conducted on leaves of vine plants in the same stage of maturation. The leaves are first treated by the Botrytis cinerea fungus for 24 hours, and then some are treated by foliar spraying of the culture filtrate or the strain according to the invention.

The results that are obtained after 24 hours are shown in FIGS. 1a (control without treatment), 1b (leaves treated with Botrytis cinerea, and then the strain according to the invention), and 1c (leaves treated with Botrytis cinerea). The leaves of vine plants that are brought into contact with Botrytis cinerea and then treated with the strain according to the invention (FIG. 1b) are greener and do not have spots relative to those that are not treated (FIG. 1c).

4) Activation of Natural Defense Systems of Plants

The objective of this study is to evaluate the capacity of the strain according to the invention, the culture filtrate, and its active compounds, to activate natural defense genes of plants, in particular Arabidopsis thaliana.

The operating procedure is described below.

The Arabidopsis thaliana plantlets are pretreated at the level of the roots by I-3639, a mutant strain, or by two of its active compounds, and then they are inoculated again on the agar Gamgorg B5 culture medium that is already inundated by a suspension of spores of Sclerotium rolfsii (10⁵ ufc/ml). The unit is incubated in a culture chamber at 25° C.

The reading of the results is carried out after one week.

It is noted that the treatment of Arabidopsis thaliana plantlets by the strain and by its active compounds activates the expression of genes linked to the defense of the plants.

The expression of the Arabidopsis thaliana defense genes is measured by using the RT-PCR technique. The defense genes of the plants that are studied are PR1 (gene that is responsible for the synthesis of antifungal molecules of plants), PDF1.2a (gene that is responsible for the activation of the ethylene and jasmonate methods, two important methods for the defense of plants), and PAL1.

The RT-PCR is done on cultivated plantlets for 10 days. It is based on the extraction of their RNA that is then converted into complementary DNA (cDNA) by using the Superscript TM II RNAse H-Reverse Transcriptase Kit (Invitrogen, Carlsbad, USA) with oligo(dT)22.

The cDNA samples that are obtained are amplified in a mixture of PCR products.

The specific primers that are used are:
For PR1: PR1 f of the sequence SEQ No. 4: 5′-CTG GCT ATT CTC GAT TTT TAA TCG-3′, PR1 r of the sequence SEQ No. 5: 5′-TCC TGC ATA TGA TGC TCC TTA TTG-3′; except for product with a dimension of 562 pb,
For PDF1.2a: PDF1 f of the sequence SEQ No. 6: 5′-TCA TGG CTA AGT TTG CTT CCA TCA TCA CCC-3′, PDF1 r of the sequence SEQ No. 7: -5′ GTA GAT TTA ACA TGG GAC TGC GAC-3′; except for the product with a dimension of 252 pb,
For PAL1: PAL1 f of the sequence SEQ No. 8: 5′-CGG AGG AGG AGT GGA CGC TAT-3′, PAL1 r of the sequence SEQ No. 9: 5′-TGC GAC ACC GTT TTT GGT TCT-34; except for the product with a dimension of 378 pb.

The PCR control is produced by the use of the cleaning gene EF1 A4 (Liboz et al., 1990): EF1 f of the sequence SEQ No. 10: 5′-ATG CCC CAG GAC ATC GTG ATT TCA-3′, EF1 r of the sequence SEQ No. 11: ‘-TTG GCG GCA CCC TTA GCT GGA TCA-3’; except for the product with a dimension of 708 pb.

The thermal conditions of the cycles contain an initial denaturation stage at 94° C. for 2 minutes, followed by 34 cycles for PRE PDF1.2a and PALE or by 26 cycles for EF1 A4, 94° C., 30 seconds, 52° C., 30 seconds, 72° C., 30 seconds, and ends at 72° C. for 10 minutes.

The PCR product is loaded on electrophoresis gel and then visualized under UV by ethidum bromide fluorescence.

It is noted that the treatment of Arabidopsis thaliana plantlets by the strain and by its active compounds activates the expression of genes linked to the defense of the plants.

The results of experiments of the RT-PCR show that the beta-vulgaris strain according to the invention activates the expression of the defense genes of the plants PR1 (gene that is responsible for the synthesis of the antifungal molecules for the plants) and PDF1.2a (gene that is responsible for the activation of the ethylene and jasmonate methods, two important methods for the defense of plants).

The activation of these genes PR1 and PDF1.2a by the strain according to the invention leads to the production of:
Plant antibiotics, phytoalexins,
Defense proteins, PR proteins, and
Compounds that are intended to reinforce the walls of plant cells.

1. Streptomyces beta-vulgaris strain deposited at the CNCM under number I-3639 and at the LGM under number P-23735, characterized by a sequence of DNAr 16S SEQ No. 1.

2. A method for obtaining a culture filtrate, comprising fermentation in a liquid medium of the strain according to claim 1, or a mutant strain thereof, centrifuging and filtration.

3. A method for obtaining active compounds, comprising fermentation in a liquid medium of the strain according to claim 1, or a mutant strain thereof, centrifuging, filtration and purification.

4. A method for stimulating the growth of plants, comprising administering to the plants an effective amount of the strain according to claim 1, a mutant strain thereof, a culture filtrate obtained from the strain or a mutant strain thereof, or an active compound obtain from the strain or a mutant strain thereof.

5. A method for stimulating the root portion of the plants comprising administering to the root portions an effective amount of the strain according to claim 1, a mutant strain thereof, a culture filtrate obtained from the strain or a mutant strain thereof, or an active compound obtain from the strain or a mutant strain thereof.

6. A method for the protection of plants and the biological struggle against phytopathogenic agents, comprising administering to the plants an effective amount of an antibacterial agent comprising the strain according to claim 1, a mutant strain thereof, a culture filtrate obtained from the strain or a mutant strain thereof, or an active compound obtain from the strain or a mutant strain thereof.

7. The method according to claim 6, wherein the protection of plants is against gram-positive bacteria.

8. A method for the protection of plants and the biological struggle against phytopathogenic agents, comprising administering to the plants an effective amount of an antifungal agent comprising the strain according to claim 1, a mutant strain thereof, a culture filtrate obtained from the strain or a mutant strain thereof, or an active compound obtain from the strain or a mutant strain thereof.

9. The method according to claim 8, wherein the protection of plants is against Botrytis cinerea.

10. A method for activating the natural defense systems of plants, comprising administering to the plants an effective amount of the strain according to claim 1, a mutant strain thereof, a culture filtrate obtained from the strain or a mutant strain thereof, or an active compound obtain from the strain or a mutant strain thereof.

11. The method according to claim 10, wherein the expression of the defense genes of plants PR1 and PDF1.2a is stimulated.

12. A method for improving the assimilation of nutrients by the plants comprising administering to the plants an effective amount of the strain according to claim 1, a mutant strain thereof, a culture filtrate obtained from the strain or a mutant strain thereof, or an active compound obtain from the strain or a mutant strain thereof.

13. The method according to claim 12, wherein the assimilation by the plants of potassium, zinc, boron, calcium, copper, phosphorus, magnesium, iron, manganese, and nitrogen is improved.
